(19) United States
(12) Patent Application Publication
(10) Pub. No.: US 2006/0287606 A1
(43) Pub. Date: Dec. 21, 2006

(54) Method for detecting heart rate and systems thereof

(76) Inventors: Di-Ann Hong, Inverness, IL (US); Liliana Grajales, Bloomingdale, IL (US); Magdi A. Mohamed, Schaumburg, IL (US); Weimin Xiao, Hoffman Estates, IL (US)

Correspondence Address: MOTOROLA, INC., 1303 EAST ALGONQUIN ROAD, IL01/3RD, SCHAUMBURG, IL, 60196, US

(21) Appl. No.: 11/157,640

(22) Filed: Jun. 21, 2005

(51) Int. Cl. A61B 5/02

(52) U.S. Cl. 600/528; 600/519

(57) ABSTRACT

A method for detecting heart rate, the method including the steps of: receiving (100) a heart activity signal at an input of a Q-filter processor that includes at least a first and a second Q-filter; removing noise (300) from the heart activity signal using the Q-filter processor to generate a filtered heart activity signal; detecting (400) a heart activity pattern, that includes at least a first S1 or QRS at a first time and a second S1 or QRS at a second time, from the filtered heart activity signal; and determining (500) a heart rate value based on the time interval between the first S1 and the second S1 or between the first QRS and the second QRS.

### CROSS-REFERENCE TO RELATED APPLICATIONS

The present application is related to the following U.S. application commonly owned together with this application by Motorola, Inc.:

Ser. No. 10/854836, filed May 27, 2004, titled “Method and Apparatus for Digital Signal Filtering” by Mohamed, et al. (attorney docket no. CML01424T).

### FIELD OF THE INVENTION

This invention, in general, relates to methods for monitoring bio-signals and systems thereof. More particularly, this invention relates to methods for heart rate detection using wearable sensors employing adaptive filtering techniques.

### BACKGROUND OF THE INVENTION

Analysis of the rhythm of a heart (e.g., heart rate and Heart Rate Variability (HRV)) is one of the most important physiological indicators of human health. Heart Rate Variability is the beat-to-beat fluctuations that occur around a person's average heart rate. Aside from using heart rate information to determine a person's activity level during exercises, continuous heart rate information is used to calculate the Heart Rate Variability. By evaluating HRV it is possible to assess the onset of a cardiac disorder.

The fluctuations from beat-to-beat are attributed, in part, to the nonlinear interaction between the sympathetic and parasympathetic branches of the involuntary nervous system. The sympathetic autonomic and parasympathetic autonomic nervous systems regulate, to some extent, the sinoatrial (SA) node and atrioventricular (AV) node of the heart and, thus, largely influence the control of the heart rate. These two nervous systems operate somewhat reciprocally to effect changes in the heart rate. Generally speaking, a higher HRV is what is desirable, whereas a lower HRV has been found to be a significant predictor of cardiac mortality and morbidity.

Several devices for detection of heart rate are known in the art. These known devices are primarily skin contact sensors such as, for instance, Electrocardiograms (ECGs) with disposable electrodes, chest straps with electrodes that depend on sweat for conductivity, and Stethoscopes employed by physicians during clinical examination of patients. When used for analyzing heart rate variability, known devices like the electronic stethoscopes require a patient to be in the clinical environment, wherein the patient typically rests while a physician clinically checks the heart rate and HRV of a patient. Therefore, such devices are in general unfeasible to analyze the heart rate and HRV of a moving person.

For example, one such device uses an output of an electronic stethoscope and displays sounds, such as heart and lung sounds, which a physician is hearing and stores them on a PDA. The lung and heart sounds are replayed along with a waveform visualization in the time or frequency domain, since waveform displays reveal diagnostic information often not heard on the auscultation. This device uses a simple phonocardiogram analysis that assumes relatively noise-free heart sound signals, wherein heart rate can be detected in real time in a motion free setting, for instance while sitting at the doctor's office. Therefore, this device is not suitable for noisy signals, for example, from wearable sensors carried by a moving person.

It is also known in the art to employ band-pass filters, FFT implementations, and peak-detection methods in analyzing heart sound or ECG signals. However, these analysis approaches have difficulty in accurately determining a heart rate based on noisy signals from wearable sensors such as acoustic, optical, or electrode sensors that may be carried by a moving person.

Therefore, there exists a need to develop a computationally efficient method for detecting heart rate during situations when a person is in motion.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 is a block diagram illustrating apparatus for detecting heart rate in accordance with embodiments of the present invention;

FIG. 2 is a block diagram illustrating a dual Q-filter processor in accordance with embodiments of the present invention;

FIG. 3 is a block diagram further illustrating a Q-filter in accordance with embodiments of the present invention;

FIG. 4 is block diagram further illustrating a Q-filter in accordance with embodiments of the present invention;

FIG. 5 is a flow diagram illustrating a method for Q-filter processing in accordance with embodiments of the present invention;

FIG. 6 illustrates a threshold matrix used in the Q-filter processing in accordance with embodiments of the present invention;

FIG. 7 is a block diagram illustrating a method for S1-S2 pattern recognition for use in the apparatus of FIG. 1;

FIG. 8 is flow diagram further illustrating the method for S1-S2 pattern recognition of FIG. 7;

FIG. 9 illustrates an ECG waveform from a heart; and

FIG. 10 further illustrates the ECG waveform of FIG. 9.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail embodiments that are in accordance with the present invention, it should be observed that the embodiments reside primarily in combinations of method steps and apparatus components related to a heart rate detection system and method. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Thus, it will be appreciated that for simplicity and clarity of illustration, common and well-understood elements that are useful or necessary in a commercially feasible embodiment may not be depicted in order to facilitate a less obstructed view of these various embodiments.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

It will be appreciated that embodiments of the invention described herein may be comprised of one or more conventional processors and unique stored program instructions that control the one or more processors to implement, in conjunction with certain non-processor circuits, some, most, or all of the functions of the heart rate detection system and method described herein. The non-processor circuits may include, but are not limited to, a radio receiver, a radio transmitter, signal drivers, clock circuits, power source circuits, and user input devices. As such, these functions may be interpreted as steps of a method to perform the heart rate detection system and method described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, methods and means for these functions have been described herein. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

Generally speaking, pursuant to the various embodiments of the present invention a method and apparatus is provided for obtaining heart rate information by monitoring heart activity signals and by pre-processing the signals using a plurality (e.g., at least two) of signal processing filters, which are configured to remove noise and extract peak heart activity pattern of the signals, and by further processing these heart activity signals using signal recognition apparatus for robust heart rate detection.

Q-filters and heart activity pattern recognition methodologies are integrated for heart rate detection using portable non-skin contact sensors. More specifically, an adjustable signal pre-processing filter is utilized comprising at least two cascaded Q-filters, Q1 and Q2. Filter Q1 will have a smaller window size to filter out noise in the input signals, and filter Q2 will have a relatively larger window size mainly to extract a dual-peak heart activity pattern. The kernel parameters of these Q-filters may be automatically determined using optimization methodologies.

A Q-filter is an adaptive technique that can perform a continuum of nonlinear filtering operations. It is modeled by a unique mathematical structure, utilizing a function called the Q-measure, defined using a set of adjustable kernel parameters. The Q-filter enables efficient hardware and software implementations of a variety of useful filtering operations. One of the distinctive characteristic of the Q-filter is its low computational complexity, which makes it appropriate for intelligent applications running on low-power and small-size devices. For instance, a single Q-filter hardware accelerator may be used to perform different filtering operations. Q-filters enable efficient implementation of computationally intensive applications on embedded devices. The behavior of the Q-filter is determined by its window size n and kernel parameters k and {fⁱ}. For a given λ, corresponding density generator values {fⁱ}, and the parameter window size n, the Q-filter can be trained, for instance off-line, using optimization methodologies to estimate these parameters. The parameters obtained from the off-line training may then be used for the on-line data processing.

After the heart activity signal is pre-processed by the cascade of Q-filters, the signal is further processed by a heart signal recognition methodology. For example, the physiologic heart sound signal is characterized by a normal first heart sound (S1) and a normal second heart sound (S2). S1 is of longer duration and lower pitch, and S2 is of shorter duration and higher pitch. These exemplary characteristics of a heart sound signal may serve as the basis for heart sound recognition methods in accordance with embodiments of the present invention. Moreover, in accordance with embodiments of the present invention, an acoustic device may be constructed with the intelligence to implement a real-time feature analysis to determine accurate heart rate from extremely noisy input signals. Moreover, the teachings in accordance with the present invention can also be expanded to the analysis of heart auscultation and phonocardiogram in the diagnosis of heart disease, and to applications of computerized respiratory sound analysis.

Those skilled in the art will realize that the above recognized advantages and other advantages described herein are merely exemplary and are not meant to be a complete rendering of all of the advantages of the various embodiments of the present invention.

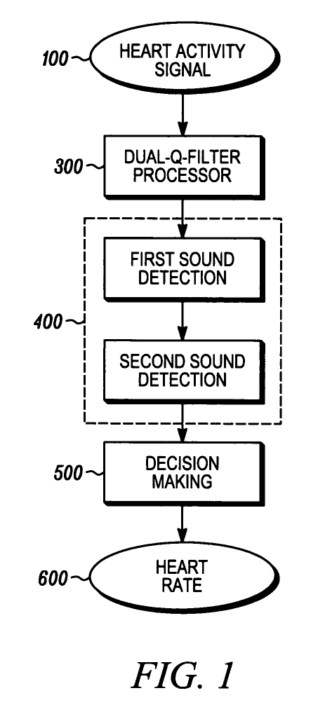

Referring now to the drawings, and in particular FIG. 1, a system and a corresponding method for measuring heart rate is shown and indicated generally at blocks 300-500. Those skilled in the art, however, will recognize and appreciate that the specifics of this illustrative example are not specifics of the invention itself and that the teachings set forth herein are applicable in a variety of alternative settings. For example, the system is described below by reference to its use in acoustic sensor apparatus. However, it should be well understood by those of ordinary skill in the art that the description is equally applicable to the system as used, for instance, in electrode sensor apparatus. Moreover, it should be further understood by those skilled in the art that the system of FIG. 1 may comprise an acoustic sensor system, an electrical sensor system, an optical sensor system, a portable sensor system, a wearable portable sensor system, and/or a wireless sensor system.

Accordingly, the system illustrated in FIG. 1 for measuring heart rate may, for example, be included in a wearable sensor device, such as, an acoustic sensor system for heart rate detection or an electrode sensor system for heart rate detection. As illustrated in FIG. 1, the system for measuring heart rate may include a Q-filter processor 300 that in this illustration is a dual Q-filter processor, a S1-S2 (or QRS-T) pattern recognition apparatus 400 that in this illustrations is a dual peak (e.g., dual sound) heart pattern recognition apparatus, and decision making apparatus 500. In operation, a heart activity signal 100 (or a pre-filtered version thereof) may be input into the Q-filter processor 300, and a heart rate measurement 600 may be generated at an output of the decision making apparatus 500, upon processing of the heart activity signal 100 by the system blocks 300-500. Moreover, a general method in accordance with embodiments of the present invention for detecting heart rate comprises the steps of: receiving (100) a heart activity signal at an input of a Q-filter processor that includes at least a first and a second Q-filter; removing noise (300) from the heart activity signal using the Q-filter processor to generate a filtered heart activity signal; detecting (400) a heart activity pattern, that includes at least a first S1 or QRS at a first time and a second S1 or QRS at a second time, from the filtered heart activity signal; and determining (500) a heart rate value based on the time interval between the first S1 and the second S1 or between the first QRS and the second QRS.

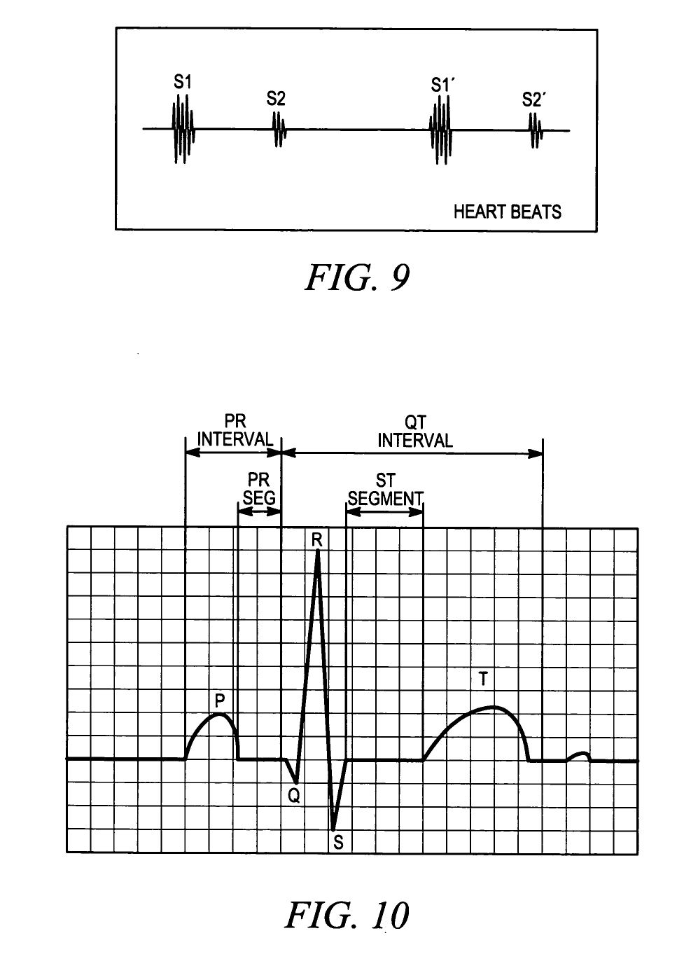

In one implementation, the heart activity signal (S) 100 may be characterized by a first heart sound (S1) and a second heart sound (S2), as in FIG. 9. Usually, S1 and S2 each have a frequency that is in the range of 20 to 70 Hz. S1 is typically of longer duration and of lower pitch than S2. Those skilled in the art will realize that in another implementation, the heart activity signal may be characterized by a heart electrical pattern, as in FIG. 10, comprising a QRS signal and a T wave signal, and that the principles herein are equally applicable to the latter heart activity signal characterization.

In one embodiment, the heart activity signal 100 may undergo processing by pre-filtering techniques and apparatus as is well known in the art such as, for instance, using analog pre-filtering techniques and apparatus. The pre-filtering may, for instance, be used for spike attenuation of the received heart activity signal. The heart activity signal or the pre-filtered heart activity signal is then passed to the dual Q-filter processor 300, which filters out noise in the heart signal 100 to extract a more clear heart sound. The noise may comprise, for instance, ambient noise in the environment and noise resulting from movement by the wearer of the sensor system that embodies the present invention. This clearer heart sound is further processed to get a heart activity (e.g., sound) pattern using the S1-S2 pattern recognition apparatus 400. Using the decision making apparatus 500, a cycle of the heart sound pattern may be detected, which may then be used to find the heart rate 600 and also the heart rate variability.

Thus, after rectification and moving integration pre-processing the amplitude of S1 is typically greater than the amplitude of S2, in the heart sound signal for example. This characteristic of heart sound may serve as the basis for the automated determination of the heart rate, wherein the relative amplitude of S1 and S2, and the time interval between S1 and S2 may be used to identify their coupling characteristics as explained in detail below. Moreover in another implementation, after rectification and moving integration pre-processing, the amplitude of the QRS signal is typically greater than the amplitude of the T wave in the heart electrical signal. This characteristic of heart sound may serve as the basis for the automated determination of the heart rate, wherein the relative amplitude of the QRS signal and the T wave, and the time interval between the QRS signal and the T wave may be used to identify their coupling characteristics as detailed below. In addition, the principles of the present invention may be applied to detect only the S1 or only the QRS and to determine heart rate based on the relative time interval between two S1 or two QRS.

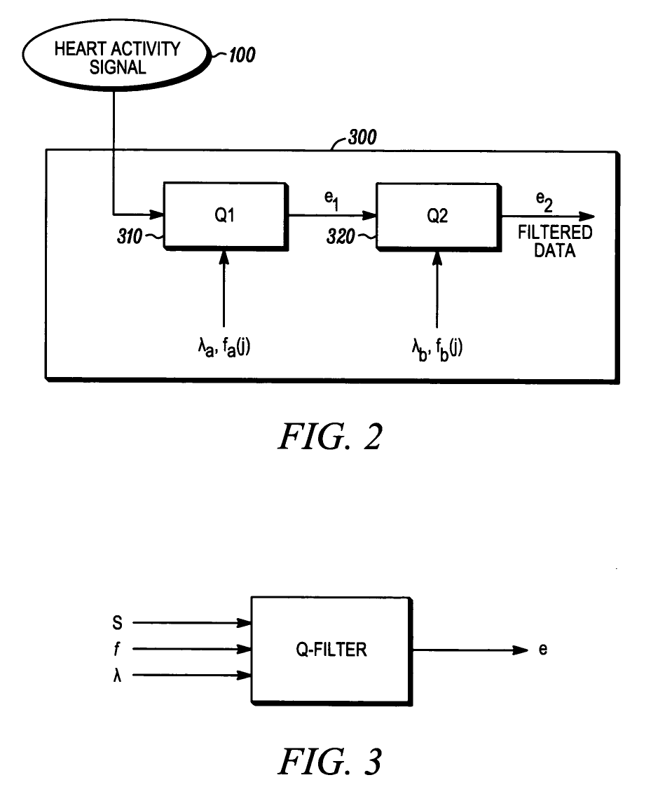

Turning now to FIG. 2, a dual Q-filter processor is shown and generally indicated at 300. In this exemplary Q-filter processor, there are two Q-filters. However, those skilled in the art will realize that in other embodiments additional Q-filters may comprise the Q-filter processor without departing from the teachings herein. The dual Q-filter processor 300 includes an input for receiving, e.g., a spike attenuated heart signal and further includes a dual Q-filter that typically comprises two cascaded filters, namely a Q1-filter 310 and a Q2-filter 320. The Q1-filter 310 may be designed with a smaller window size to filters out noise in the spike attenuated heart signal, and the Q2-filter 320 may be designed with a larger window size to extract a heart activity pattern, e.g., a dual-peak heart sound pattern. For example, in one implementation, Q-filter 310 may have a window size of n=5, and Q2-filter 320 may have a window size of n=45. The Q-filters Q1 and Q2 are serially coupled in this embodiment, although it should be realized by skilled artisans that in another embodiment, Q1 and Q2 may be coupled in parallel. Moreover, for a Q-filter processor, the coupling between separate Q-filters may be a combination of serial and parallel connections, for instance where there are more than two Q-filters in the Q-filter processor.

The Q-filter is a class of nonlinear filters that is defined as a Choquet integral with respect to a q-measure over a window of observations. By adjusting a plurality of Q-filter kernel parameters, a single Q-filter can reshape an input signal that may require the application of many different other linear and nonlinear filters.

For an input signal window S={s₁, s₂, . . . , sₙ}, where n is the kernel window size and the input values are sⱼ, for j=1, . . . n, a basic Q-filter can be constructed using the Choquet integral using the following steps.
Set a value for the filter kernel parameter λ ε [−1, ∞).
Set an n-point density generator vector f={f¹, f², . . . , fⁿ} for the input signal window S={s₁, s₂, . . . , sₙ}, where f^{j} ε [0,1], j=1, . . . , n.
Set an m point resolution vector R={r₀, r₁, . . . , rₘ₋₁}, where r₀<r₁< . . . <rₘ₋₁, for the input signal S={s₁, s₂, . . . , sₙ} with the range of signal strength, i.e., for all sⱼ, rₘᵢₙ=r₀≦sⱼ≦rₘ₋₁=rₘₐₓ.
Set an (m−1) by n threshold matrix H={hᵢⱼ} where hᵢⱼ=1 if sⱼ≧rᵢ, otherwise hᵢⱼ=0.
Calculate the q-measure based on the threshold matrix H={hᵢⱼ}, and the density generator values, f^{j}=f({xⱼ}), j=1, . . . n, as follows:

$$\begin{matrix}F = {\prod }_{j = 1}^{n} ( 1 + λ {f}^{j} ) - 1 & ( 1 a ) \\ {q}_{i} = \frac{{\prod }_{j = 1}^{n} ( 1 + λ {h}_{ij} {f}^{j} ) - 1}{F} . & ( 1 b )\end{matrix}$$

In the above equations, λ ε [−1, ∞), and λ≠0.

When λ=0, we have:

$$\begin{matrix}F = {\sum }_{j = 1}^{n} {f}^{j} & ( 2 a ) \\ {q}_{i} = \frac{{\sum }_{j = 1}^{n} {h}_{ij} {f}^{j}}{F} . & ( 2 b )\end{matrix}$$

Calculate the Choquet integral by:

$$C = {\sum }_{i = 1}^{m - 1} {q}_{i} \frac{{r}_{max} - {r}_{min}}{m - 1} = \frac{{r}_{max} - {r}_{min}}{m - 1} {\sum }_{i = 1}^{m - 1} {q}_{i} . \qquad ( 3 )$$

The filtered signal value corresponding to the input window is then
e=rₘᵢₙ+C. (4)

Turning now to FIG. 3 a block diagram of an exemplary Q-filter (e.g., Q1) is shown and generally indicated. The block diagram in FIG. 3 is likewise applicable to Q-filter Q2 of FIG. 2 except that the input signal S would in this illustration be replaced by the estimated value e₁ from the output of Q1. As shown, the Q-filter comprises an input receiving a signal S to be filtered, an input receiving variable parameter λ, and an input receiving a density generator vector f={f¹, f², . . . , fⁿ}. Accordingly by reference again to FIG. 2, Q1 has inputs S (100), λₐ, fₐ(j) and an output e₁, and Q2 has inputs e₁, λ_{b}, f_{b}(j) and an output e₂.

Returning again to FIG. 3, during operation the input signal window S={s₁, s₂, . . . , sₙ}, is input into the Q-Filter 300 with e being the output computed as an expected value of the given input. More particularly, during operation the Q-filter utilizes logic circuitry (e.g., a microprocessor controller) and memory components to construct a q-measure based on the variable parameter λ and vector f, and outputs a filtered signal based on the q-measure. The above method is also suitable for hardware implementation since the basic mathematical operations are thresholding, addition and multiplication for discrete valued input signals that can be quantized to have values between 0 and m−1. The Q-filter operation can, thus, be further simplified in such cases to enable efficient hardware implementation.

A Q-filter can be constructed using threshold decomposition and a q-measure as follows. Let S be a moving window over an input signal, that is S(t)={s₁, s₂, . . . , sₙ}, where n is the window size and the window elements are denoted by sⱼ ε {0, 1, . . . , m−1}, j=1, . . . , n, at time slot t ε Z. Form the threshold binary signals s⁽¹⁾, s^{(m-1)} by

$${s}_{j}^{( i )} = \{ \begin{matrix}1 & if {s}_{j} \geq i \\ 0 & otherwise\end{matrix} \qquad ( 5 )$$

The output of filtering the i^{th} threshold signal s⁽ⁱ⁾ at point t is defined by
Aᵢ={xⱼ|sⱼ⁽ⁱ⁾=1, j=1, . . . , n} (6a)
e⁽ⁱ⁾(t)=q(Aᵢ) (6b)
where sⱼ⁽ⁱ⁾=1, . . . n, are Boolean variables defining the crisp set Aᵢ, the argument of the q-measure q(.) defined using a kernel of size n. The output of the Q-filter with respect to q(.) at point t is now:

$$e ( t ) = {\sum }_{i = 1}^{m - 1} {ⅇ}^{( ⅈ )} ( t ) \qquad ( 7 )$$

where the values e⁽ⁱ⁾(t) of the q-measure are real values in the unit interval [0,1].

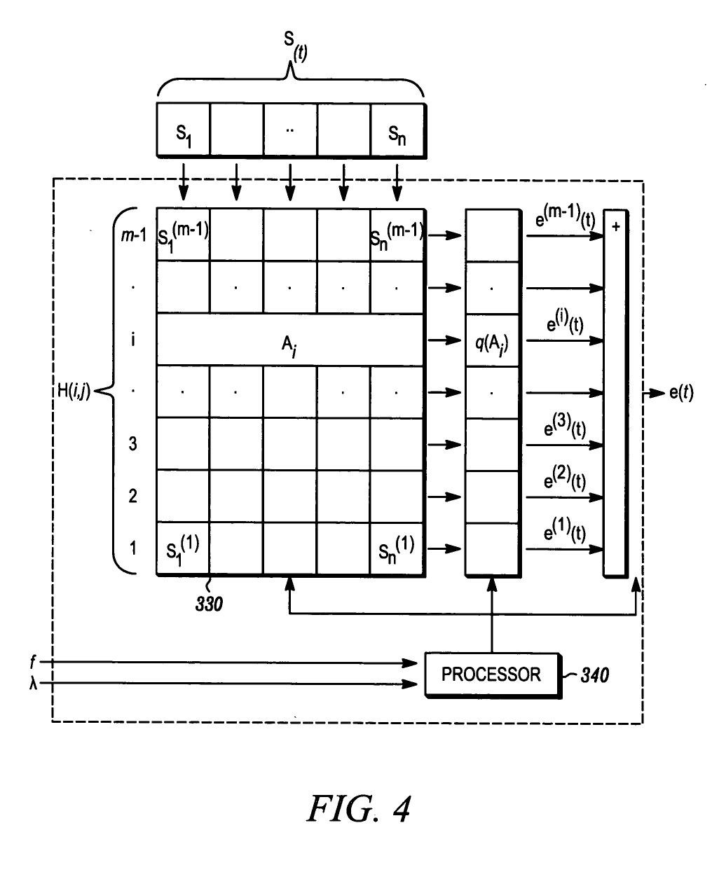

The above procedure is illustrated in FIG. 4, which shows a more-detailed block diagram of the Q-filter illustrated in FIG. 3. As shown, an input signal S(t) enters filter 300 and enters thresholder 330. Processor 340 instructs thresholder 330 to Form the m−1 threshold binary signals s⁽¹⁾, . . . , s^{(m-1)} as described above in equation (5), where Aᵢ={xⱼ|sⱼ⁽ⁱ⁾=1, j=1, . . . , n} is a crisp set obtained by thresholding the input signal S(t) at threshold value i. Thresholder 330 outputs Aᵢ and processor 340 constructs e⁽ⁱ⁾(t) for each threshold value by computing the q-measure of the crisp set Aᵢ. More particularly e⁽ⁱ⁾(t)=q(Aᵢ) as defined in equation (6b). The values for e⁽ⁱ⁾(t) are summed to produce e(t).

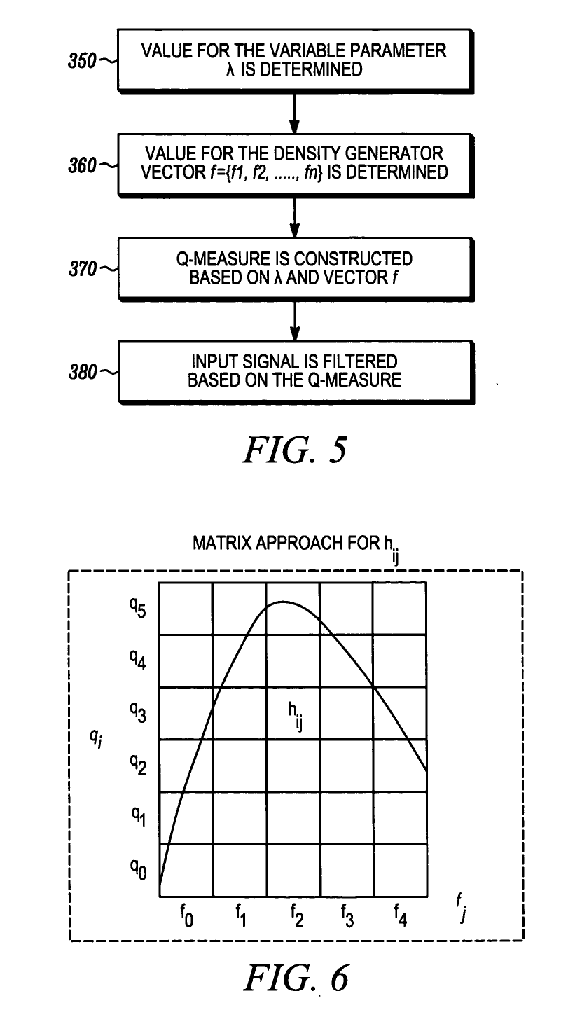

Turning now to FIG. 5 a flow diagram showing operation of the Q-filter of FIG. 3 is shown and generally indicated. The logic flow begins at step 350 where a value for variable parameter λ is determined. At step 360, a value for the density generator vector f={f¹, f², . . . , fⁿ} is determined and a q-measure is constructed based on λ and vector f (step 370). In one embodiment, parameters λ and f are determined off-line pursuant to design constraints of the given Q-filter. Finally, at step 380 an input signal is filtered based on the q-measure.

Turning now to FIG. 6 a matrix approach for hᵢⱼ in accordance with embodiments of the present invention is shown and generally indicated, wherein the set of density generator values depends on the size of the window used in the filter and wherein each square, hᵢⱼ, in the matrix may correspond, for instance, to a discreet value, e.g., 0 if the square is above the curve and 1 if the square is below the curve.

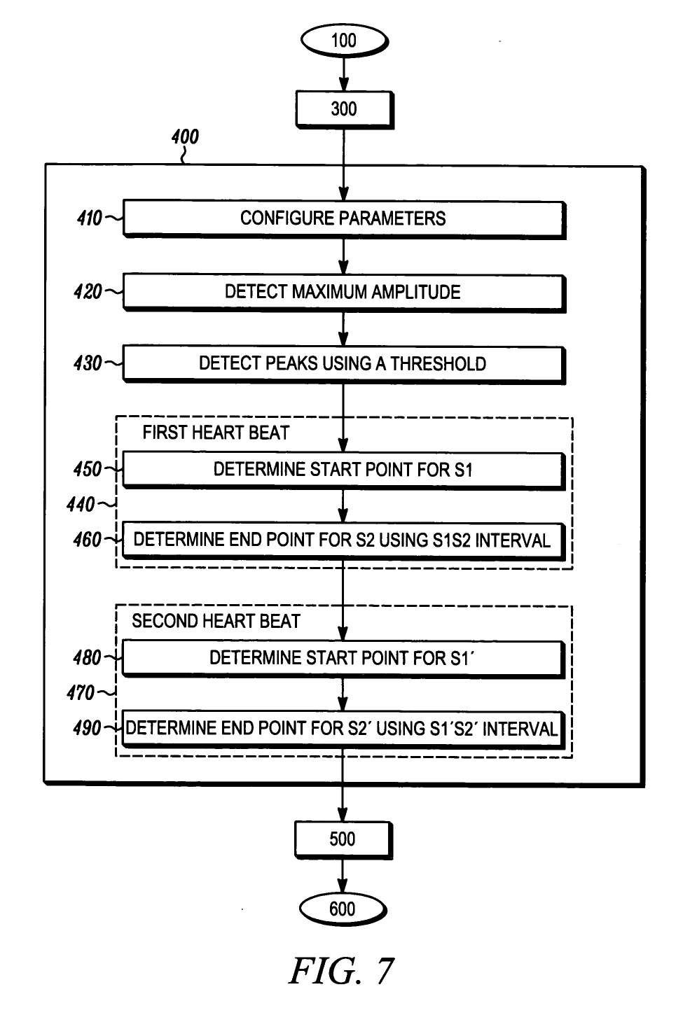

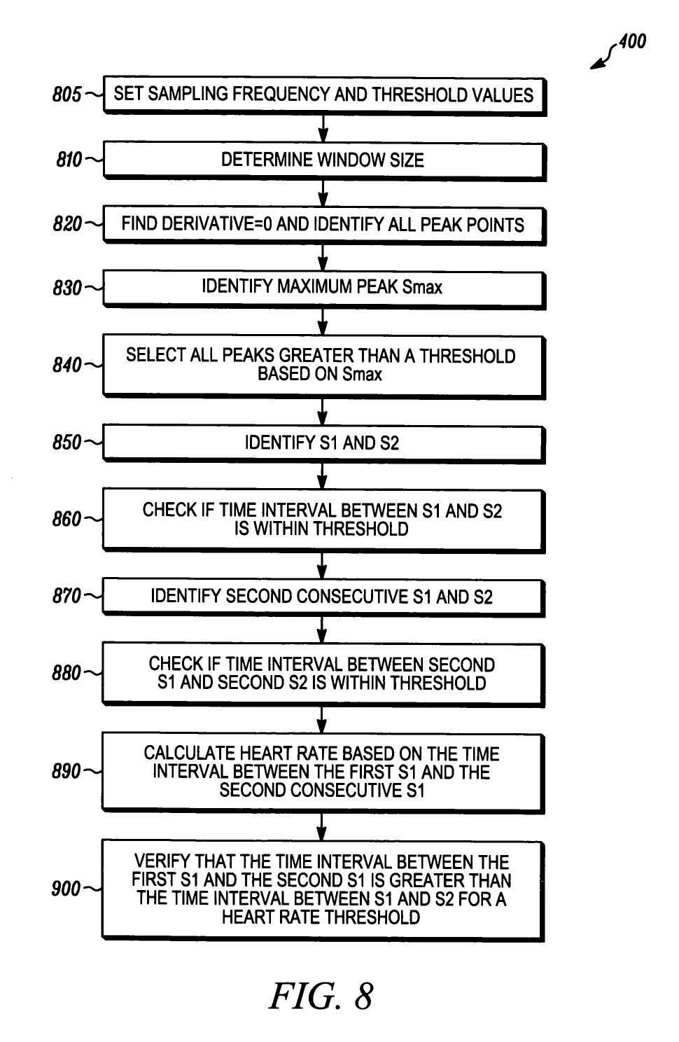

The following nomenclature is applicable to FIGS. 7 and 8:
fs, sampling frequency;
N, frame window size (e.g., total points of input data);
S(i), amplitude of input data at i point, i=1, 2, . . . , N;
PN, total number of peaks found in the input data window, which meet the amplitude threshold;
P(j), a point in 1 to N corresponding to the jth peak point in the window, wherein P(j) is a subset of i=1, 2, . . . , N; j=1, 2, . . . , PN;
Smax, the maximal amplitude of the S(i) series;
k, a threshold factor for the amplitude of the first heart sound S1 and the second heart sound S2 in the window;
Interval_min, the minimal interval (points) between S1 and S2;
Interval_max, the maximal interval (points) between S1 and S2;
HR_norm, the heart rate value for which the diastole time is always greater than the systole time; and
HR_high, the heart rate value for which the diastole time is equal or possibly less than the systole time.

The output of dual-Q-filter 300 (e.g., the filtered signal) is further processed by pattern recognition apparatus (and corresponding methodology) 400 of FIG. 1. Turning now to FIG. 7 a flow diagram of an exemplary of S1 and S2 pattern recognition methodology for heart rate detection is shown and generally indicated. This flow diagram explains the S1-S2 coupling characteristics mentioned above.

At step 410, at least one parameter is configured, including setting a sampling frequency (e.g., fs) and at least one threshold value (e.g., Interval_min, Interval_max) and determining a window size, e.g., N. The filtered signal is sampled in an open window using the predetermined sampling frequency fs which gives the amplitude of the input signal, S(i) where i=1, 2, . . . N. The maximum amplitude of the input signal, Smax, is then identified at 420. At step 430, all peaks that meets an amplitude threshold, k, that is based on Smax (e.g., k*Smax) is selected. These peak points are represented by p(j) where j=1, 2, . . . PN, and wherein PN represents the number of these peaks, and the values of these peaks can be denoted as S(p(j)).

The first heart beat, e.g. characterized by at least S1 and that may also be characterized by S2, may be detected at step 440. Accordingly, the first S1 may be identified (450), starting from j=1 such that S(p(j)) >S(p(j+1)). Then the first S2 may be identified (460) using the predetermined interval_min and interval_max between S1 and S2, wherein the time interval between S1 and S2 ideally falls between interval_min and interval_max. In this illustration, S1 and S2 comprise the first heart beat. In a similar manner, a second heart beat may be detected at step 470 by at least determining a second S1, e.g., S1′, (480) and in this illustration a second S2, e.g., S2′, (490), for instance using the same predetermined S1 to S2 time interval. This S1 and S2 pattern may be received into decision making apparatus 500 and a heart rate for one cycle may be calculated using, for instance, the formula Heart rate=one cycle duration*fs/60, wherein one cycle duration is, for instance, the time duration between S1 and S1′.

Turning now to FIG. 8 an exemplary methodology for S1-S2 pattern recognition is shown and generally indicated. The steps involved in the methodology are as follows. At steps 805, 810 parameters are configured. For instance, the following predetermined parameters may be set: fs=500 (Hz); k=0.6; Interval_min=125; Interval_max=175; and N=2000. For window size N=2000, determine derivatives of S(i), and find all peak points in S(i) whose first derivative is zero (820). Identify (830) the maximum value: Smax. Select (840) all peaks whose value>k*Smax. These peak points are represented by p(j) (representing the location of the peaks in the window), where j=1, 2, . . . PN, and wherein PN represents the total number of these peaks, and the values of these peaks can be denoted as S(p(j)). Search (850) for the first S1 in the window. For example, start at j=1 to find the first conjunct pair of peaks where S(P(j))>S(P(j+1)), then P(j) is the first S1 point, e.g., P(s11). Then, optionally search for the first S2 in the window, e.g., from point P(s11), the next peak is P(s11+1). If at step 860, {P(s11+1)−P(s11)}>Interval_min and {P(s11+1)−P(s11)}<Interval_max, then, the P(s11+1) is the first S2 point (e.g., P(s12)), and the P(s11) is validated as the first S1. It should be noted that steps 850 and 860 effectively form a loop for j until a valid S1 and S2 is found or j=PN.

Likewise, at step 870, in a similar manner as above with respect to steps 850 and 860 a second S1 and S2, e.g. S1′ and S2′ may be detected in the window, for instance, from P(s12). Let S1′ be P(s21) in this illustration, and let S2′ be P(s22). If both P(s11) and P(s21) are validated, the heart rate may be determined (890) by computing (P(s21)−P(s11))*fs/60 in this given window. Moreover to validate the heart rate (900), the time between S1 & S2 may be measured, e.g., [P(s21)−P(s12)]=diastole interval, and [P(s12)−P(s11)]=systole interval, whereby if [P(s21)−P(s12)]/[P(s12)-P(s11)]<1, then only when the heart rate is greater than HR_norm the resulting heart rate value is acceptable. Thereafter, another window may be opened and steps 805-900 may be repeated.

FIG. 10 illustrates an electrical signal of the heart having the well known QRS complex, and T wave of an electrocardiogram (e.g., EKG or ECG) waveform. The QRS complex represents the time it takes for depolarization of the ventricles, due to ventricular depolarization. The T wave is due to the ventricular repolarization. In this case, the heart rate pattern method comprises: receiving a first QRS signal and a first T wave signal at the input of a first Q-filter; filtering out noise from the input signals by the first Q-filter; extracting a dual-peak heart electrical signal pattern, for example, by a second Q-filter; receiving the extracted dual-peak heart electrical signal pattern by suitable signal recognition apparatus; determining a heart rate pattern by decision making apparatus by identifying the relative amplitude of QRS and T and the time interval between QRS and T and by relative measurements between a first QRS and a second QRS. The filtering and pattern recognition technique proposed for the heart sound example using S1 and S2, can be equally applied to the heart electrical signal using identified QRS and T peaks.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A method for detecting heart rate comprising the steps of:
receiving a heart sound signal at an input of a Q-filter processor that comprises at least a first and a second Q-filter;
removing noise from the heart sound signal using the Q-filter processor to generate a filtered heart sound signal;
detecting a heart sound pattern comprising at least a first S1 at a first time and a second S1 at a second time, from the filtered heart sound signal; and
determining a heart rate value based on the time interval between the first S1 and the second S1.

2. The method of claim 1, wherein the heart sound pattern is a dual-peak heart sound pattern that further comprises a first S2 at a third time between the first and second times and a second S2 at a fourth time after the second time.

3. The method of claim 1, wherein the step of detecting a heart sound pattern comprises the steps of:
configuring a set of parameters;
detecting a maximum amplitude of the filtered heart sound signal;
detecting a plurality of peaks in the filtered heart sound signal, each having an amplitude greater than a threshold value that is based on the maximum amplitude;
detecting a first heart beat from the plurality of peaks that includes the first S1; and
detecting a second heart beat from the plurality of peaks that includes the second S1.

4. The method of claim 3, wherein the step of configuring a set of parameters includes setting a sampling frequency and at least one threshold value and determining a window size.

5. The method of claim 1, wherein the step of removing noise from the heart sound signal to generate a filtered heart sound signal comprises the steps of:
determining a value of a variable parameter λ;
determining a density generator vector f={f¹, f², . . . , fⁿ};
constructing a q-measure based on the variable parameter λ and the vector f; and
filtering the heart sound signal based on the q-measure.

6. A method for detecting heart rate comprising the steps of:
receiving a heart electrical signal at an input of a Q-filter processor that comprises at least a first and a second Q-filter;
removing noise from the heart electrical signal using the Q-filter processor to generate a filtered heart electrical signal;
detecting a heart electrical pattern comprising at least a first QRS at a first time and a second QRS at a second time, from the filtered heart electrical signal; and
determining a heart rate value based on the time interval between the first QRS and the second QRS.

7. The method of claim 6, wherein the heart electrical pattern is a dual-peak heart electrical pattern that further comprises a first T at a third time between the first and second times and a second T at a fourth time after the second time.

8. The method of claim 6, wherein the step of detecting a heart electrical pattern comprises the steps of:
configuring a set of parameters;
detecting a maximum amplitude of the filtered heart electrical signal;
detecting a plurality of peaks in the filtered heart electrical signal having an amplitude greater than a threshold that is based on the maximum amplitude;
detecting a first heart beat from the plurality of peaks that includes the first QRS; and
detecting a second heart beat from the plurality of peaks that includes the second QRS.

9. The method of claim 8, wherein the step of configuring a set of parameters includes setting a sampling frequency and at least one threshold value and determining a window size.

10. The method of claim 6, wherein the step of removing ambient noise from the heart electrical signal to generate a filtered heart electrical signal comprises the steps of:
determining a value of a variable parameter λ;
determining a density generator vector f={f¹, f², . . . , fⁿ};
constructing a q-measure based on the variable parameter λ and the vector f; and
filtering the heart electrical signal based on the q-measure.

11. Apparatus for heart rate detection from a heart activity signal comprising:
a Q-filter processor comprising at least a first and a second Q-filter coupled together for receiving a heart activity signal and removing noise from the heart activity signal to generate a filtered heart activity signal;
a heart pattern recognition device for detecting a heart activity pattern comprising at least a first indicia at a first time and a second indicia at a second time, from the filtered heart activity signal; and
a decision making device for determining a heart rate value based on the time interval between the first indicia and the second indicia.

12. The apparatus of claim 11, wherein:
the first indicia is one of a first S1 and a first QRS;
the second indicia is one of a second S1 and a second QRS; and
the heart rate is based on one of the interval between the first and second S1 and the interval between the first and second QRS.

13. The apparatus of claim 11, wherein the apparatus comprises at least one of an acoustic sensor system, an electrical sensor system, and optical sensor system, a portable sensor system, a wearable portable sensor system and a wireless sensor system.

14. The apparatus of claim 11, wherein the first and second Q-filters are coupled one of in series and in parallel.

15. The apparatus of claim 11, wherein the first and second Q-filters are non-linear signal processing filters.

16. The apparatus of claim 11, wherein the first Q-filter has a first window size, and the second Q-filter has a second window size that is larger than the first window size.

17. The apparatus of claim 11, wherein the Q-filter processor further comprises at least one other Q-filter and the Q-filters are coupled together at least one of in series and in parallel.

18. The apparatus of claim 11, wherein the first and second Q-filters comprise logic circuitry and a memory coupled together to generate a q-measure based on a variable parameter λ and a vector f and to output a filtered signal based on the q-measure.
